(19) United States
(12) Patent Application Publication
(10) Pub. No.: US 2025/0221735 A1
(43) Pub. Date: Jul. 10, 2025

(54) VASCULAR PUNCTURE APPARATUS AND METHOD FOR CONTROLLING THE SAME

(71) Applicant: TERUMO KABUSHIKI KAISHA, Tokyo (JP)

(72) Inventors: Takumi FUKUDA, Hadano-shi (JP); Takito INUKAI, Isehara-shi (JP); Kazuki NAKASONE, Fujinomiya-shi (JP); Yoichiro KUWANO, Princeton, NJ (US)

(73) Assignee: TERUMO KABUSHIKI KAISHA, Tokyo (JP)

(21) Appl. No.: 19/089,957

(22) Filed: Mar. 25, 2025

Related U.S. Application Data
(63) Continuation of application No. PCT/JP2023/034446, filed on Sep. 22, 2023.

(30) Foreign Application Priority Data
Sep. 30, 2022 (JP) 2022-157385

(51) Int. Cl. A61B 17/34; A61B 17/00; A61B 90/00

(52) U.S. Cl. CPC A61B 17/3403; A61B 2017/00057; A61B 2017/00128; A61B 2017/3492; A61B 2090/061; A61B 2090/064; A61B 2090/067

(57) ABSTRACT

A vascular puncture apparatus capable of automatically performing or stopping appropriate puncture even when deviation in a position of a puncture target or tissue including the puncture target with respect to the vascular puncture apparatus or deviation in a position or inclination of the vascular puncture apparatus itself occurs, and a method for controlling the vascular puncture apparatus are provided. The vascular puncture apparatus includes a drive unit that moves a puncture needle and a control unit capable of controlling the movement of the drive unit, a detection unit that detects at least one of relative positions between the vascular puncture apparatus and a puncture target, relative positions between the vascular puncture apparatus and tissue including the puncture target, a position of the vascular puncture apparatus, and an inclination of the vascular puncture apparatus.

### CROSS-REFERENCES TO RELATED APPLICATIONS

This application is a continuation of International Application No. PCT/JP2023/034446 filed on Sep. 22, 2023, which claims priority to Japanese Patent Application No. 2022-157385 filed on Sep. 30, 2022, the entire content of both of which is incorporated herein by reference.

### TECHNOLOGICAL FIELD

The present disclosure generally relates to a vascular puncture apparatus that automatically punctures a blood vessel and a method for controlling the vascular puncture apparatus.

### BACKGROUND DISCUSSION

In order to secure an access route to a blood vessel for drug administration or endovascular treatment, vascular puncture is performed in which a human body is punctured with a puncture needle having a sharp needle tip, the puncture needle being covered by a flexible outer tube. The access route can be secured using the outer tube by inserting the outer tube into the blood vessel along with the puncture needle and then removing only the puncture needle. Since an operator cannot visually observe a blood vessel from a skin surface in the vascular puncture, the operator estimates a position of the blood vessel on the basis of standard knowledge of how blood vessels run and skills including tactile perception of vascular pulsation.

In addition, there are devices that automatically perform vascular puncture. When a puncture device performs puncture, deviation might occur due to movement of a blood vessel accompanying movement of an arm of a patient, movement of an attachment position between a puncture needle and the puncture device, or the like. When such deviation occurs, there is a possibility that it may be difficult to puncture a blood vessel as a puncture target, it may be difficult to secure an access route, or that a blood vessel may be excessively damaged by the puncture needle. Japanese Patent Application Publication No. 2019-47956 A describes a medical aid to which a puncture aid or a hemostatic aid can be attached while suppressing movement of an arm of a patient. The medical aid described in Japanese Patent Application Publication No. 2019-47956 A fixes an arm by sandwiching the arm between a base member and a fixing member.

A diameter of a target radial artery is about 2 mm to 3 mm. When the medical aid described in Japanese Patent Application Publication No. 2019-47956 A is used, unless the arm is fixed to the medical aid very strongly, a blood vessel easily moves due to deformation of tissue of the skin and the like when a patient moves the arm. In addition, when the arm is fixed to the medical aid very strongly, patient comfort can be impaired. Accordingly, it can be difficult to achieve both patient comfort and fixation of a blood vessel. Furthermore, since the medical aid described in the Japanese Patent Application Publication No. 2019-47956 A cannot detect movement of an arm or a blood vessel, when the arm or the blood vessel moves, the medical aid cannot appropriately perform puncture, and might cause unnecessary pain to a patient.

### SUMMARY

A vascular puncture apparatus is disclosed, which is capable of automatically performing or stopping appropriate puncture even when deviation in a position of a puncture target or tissue including the puncture target with respect to the vascular puncture apparatus or deviation in a position or an inclination of the vascular puncture apparatus itself occurs, and a method for controlling the vascular puncture apparatus.

(1) A vascular puncture apparatus is disclosed, which includes a fixing base including a holding unit capable of holding a part of a human body, a notification unit that notifies of information using an image and/or sound, a drive unit that moves a puncture needle, a control unit capable of controlling the movement of the drive unit, and a detection unit that detects at least one of relative positions between the vascular puncture apparatus and a puncture target, relative positions between the vascular puncture apparatus and tissue including the puncture target, a position of the vascular puncture apparatus, and an inclination of the vascular puncture apparatus, in which the control unit obtains a detection result before puncture from the detection unit before the puncture operation by the drive unit, the control unit continuously or intermittently obtains, after a start of the puncture operation by the drive unit, a detection result after the start of the puncture from the detection unit, the control unit calculates an amount of change in at least one of the relative positions between the vascular puncture apparatus and the puncture target, the relative positions between the vascular puncture apparatus and the tissue including the puncture target, the position of the vascular puncture apparatus, and the inclination of the vascular puncture apparatus by comparing the detection result after the start of the puncture with the detection result before the puncture, and the control unit stops the movement of the puncture needle by the drive unit or changes a movement path of the puncture needle if the at least one amount of change is larger than or equal to a threshold or larger than the threshold.

The vascular puncture apparatus according to (1) can automatically perform or stop appropriate puncture even when deviation in the position of the puncture target or the tissue including the puncture target with respect to the vascular puncture apparatus or deviation in a position or an inclination of the vascular puncture apparatus itself occurs.

(2) In the vascular puncture apparatus according to (1), the detection unit may include a cross-sectional image detection portion, which is an echograph that obtains a cross-sectional image, and the control unit may detect a feature point indicating a specific site from the cross-sectional image and calculate the position of the puncture target or the tissue including the puncture target. As a result, the vascular puncture apparatus can detect deviation in the position of the puncture target or the tissue including the puncture target with respect to the vascular puncture apparatus from the cross-sectional image obtained from the cross-sectional image detection portion.

(3) In the vascular puncture apparatus according to (2), the feature point may be at least one of a shape of a blood vessel, a contour of a bone, or a contour of a muscle. As a result, the vascular puncture apparatus can easily detect at least one of the shape of the blood vessel, the contour of the bone, and the contour of the muscle from the cross-sectional image and calculate the position of the puncture target or the tissue including the puncture target.

(4) In the vascular puncture apparatus according to any one of (1) to (3), the detection unit may include an image detection portion, which is a camera that obtains an image, and the control unit may detect a feature point indicating a specific site from the image and calculates the position of the tissue including the puncture target. As a result, the vascular puncture apparatus can detect deviation in the position of the puncture target or the tissue including the puncture target with respect to the vascular puncture apparatus from the image obtained from the image detection portion.

(5) In the vascular puncture apparatus according to (4), the feature point may be at least one of a shape of an arm, a position of a vein, size of a lentigo, or a position of a lentigo. As a result, the vascular puncture apparatus can easily detect at least one of the shape of the arm, the position of the vein, the size of the lentigo, and the position of the lentigo from the image as a feature point and calculate the position of the tissue including the puncture target.

(6) In the vascular puncture apparatus according to any one of (1) to (5), the detection unit may include an arm position detection portion that detects a relative distance from the vascular puncture apparatus to an object held by the holding unit, and the control unit may calculate the position of the tissue including the puncture target from the relative distance obtained from the arm position detection portion. As a result, the vascular puncture apparatus can detect deviation in the position of the tissue including the puncture target with respect to the vascular puncture apparatus from the relative distance obtained from the arm position detection portion.

(7) In the vascular puncture apparatus according to any one of (1) to (6), the drive unit may include a needle holding portion that fixes the puncture needle, the detection unit may include a puncture needle detection portion that is disposed in the needle holding portion and that detects the puncture needle, and the control unit may calculate a position of the puncture needle from a detection result obtained from the puncture needle detection portion. As a result, the vascular puncture apparatus can detect deviation in the position of the puncture needle with respect to the needle holding portion from a detection result obtained from the puncture needle detection portion.

(8) In the vascular puncture apparatus according to any one of (1) to (7), the detection unit may include a main body detection portion that is disposed in the vascular puncture apparatus and that detects an absolute position or an inclination. As a result, the vascular puncture apparatus can detect deviation in the position and inclination of the vascular puncture apparatus from a detection result obtained from the main body detection portion.

(9) In the vascular puncture apparatus according to any one of (1) to (8), the holding unit may include a plurality of pressure sensors, the detection unit may include a pressure detection portion including the plurality of pressure sensors arranged in the holding unit, and the control unit may calculate a position of the tissue including the puncture target from a detection result obtained from the pressure detection portion. As a result, the vascular puncture apparatus can detect deviation in the position of an arm, which is the tissue including the puncture target, with respect to the holding unit from the detection result obtained from the pressure detection portion.

(10) In the vascular puncture apparatus according to any one of (1) to (9), the control unit may determine, if the amount of change in the position of the tissue including the puncture target is smaller than or equal to the threshold or smaller than the threshold and the amount of change in the position of the puncture target is larger than or equal to the threshold or larger than the threshold, whether or not puncture of the puncture target is possible by controlling the drive unit and changing a movement path of the puncture needle from information regarding a position of a tip of the puncture needle and the position of the puncture target, change the movement path of the puncture needle if determining that the puncture of the puncture target is possible, and stop the movement of the puncture needle if determining that the puncture of the puncture target is impossible. As a result, the vascular puncture apparatus can determine that only the position of the blood vessel as the puncture target has moved whereas the arm has not moved with respect to the vascular puncture apparatus, change the movement path of the puncture needle if the puncture of the blood vessel is possible by changing the movement, and stop the puncture of the puncture needle if the puncture of the blood vessel is impossible by changing the movement path of the puncture needle.

(11) In the vascular puncture apparatus according to (10), the control unit may determine, if the amount of change in the position of the tissue including the puncture target is smaller than the threshold or smaller than or equal to the threshold and the amount of change in the position of the puncture target is larger than or equal to the threshold or larger than the threshold, whether or not the position of the tip of the puncture needle has reached a same depth as a rear wall of a blood vessel from the information regarding the position of the tip of the puncture needle and the position of the puncture target, change the movement path of the puncture needle if determining that the position of the tip of the puncture needle has not reached the same depth as the rear wall of the blood vessel, and stop the movement of the puncture needle if determining that the position of the tip of the puncture needle has reached the same depth as the rear wall of the blood vessel. As a result, if determining that only the position of the blood vessel as the puncture target has moved whereas the arm has not moved with respect to the vascular puncture apparatus, the vascular puncture apparatus can change the movement path while assuming that the puncture of the blood vessel is possible by changing the movement path of the puncture needle if the position of the tip of the puncture needle has not reached the same depth as the rear wall of the blood vessel, and stop the puncture with the puncture needle while assuming that the puncture of the blood vessel is impossible by changing the movement path of the puncture needle if the position of the tip of the puncture needle has reached the same depth as the rear wall of the blood vessel.

(12) In the vascular puncture apparatus according to any one of (1) to (11), the control unit may determine, if the amount of change is larger than or equal to the threshold or larger than the threshold, whether or not the tip of the puncture needle is in a body, and the control unit may stop the movement of the puncture needle by the drive unit if determining that the tip of the puncture needle is in the body, and the control unit may stop, if determining that the tip of the puncture needle is not in the body, the movement of the puncture needle by the drive unit and return the puncture needle to an original position. As a result, if the tip of the puncture needle is in the body, the vascular puncture apparatus can stop the puncture needle in order to wait for a determination by the operator. Furthermore, if the tip of the puncture needle is outside the body, the vascular puncture apparatus can suppress erroneous puncture by returning the puncture needle to the original position, and improve workability thereafter.

(13) The vascular puncture apparatus according to any one of (1) to (12), the control unit may calculate the amount of change in the position of the puncture target or the tissue including the puncture target or the amount of change in the position of the vascular puncture apparatus by comparing detection results obtained by the detection unit at any two points in time after the start of the puncture operation by the drive unit, and the control unit may stop the movement of the puncture needle by the drive unit or change the movement path of the puncture needle if the amount of change is larger than or equal to the threshold or larger than the threshold. As a result, the vascular puncture apparatus can detect deviation in the position of the puncture target or the tissue including the puncture target and deviation in the position of the vascular puncture apparatus, which occur after the start of the puncture operation, and automatically perform or stop appropriate puncture.

(14) A method is disclosed for controlling a vascular puncture apparatus that includes a fixing base including a holding unit capable of holding a part of a human body, a notification unit that notifies of information using an image and/or sound, a drive unit that moves a puncture needle, and a control unit capable of controlling the movement of the drive unit, the method including the steps of obtaining, before a puncture operation by the drive unit, a detection result before puncture using a detection unit capable of detecting at least one of relative positions between the vascular puncture apparatus and a puncture target, relative positions between the vascular puncture apparatus and tissue including the puncture target, a position of the vascular puncture apparatus, and an inclination of the vascular puncture apparatus, continuously or intermittently obtaining, after a start of the puncture operation by the drive unit, a detection result after the start of the puncture from the detection unit, calculating an amount of change in at least one of the relative positions between the vascular puncture apparatus and the puncture target, the relative positions between the vascular puncture apparatus and the tissue including the puncture target, the position of the vascular puncture apparatus, and the inclination of the vascular puncture apparatus by comparing the detection result after the start of the puncture with the detection result before the puncture, and stopping the movement of the puncture needle by the drive unit or changes a movement path of the puncture needle if the at least one amount of change is larger than or equal to a threshold or larger than the threshold.

The method for controlling a vascular puncture apparatus according to (11) can automatically perform or stop appropriate puncture even when deviation in the position of the puncture target or the tissue including the puncture target with respect to the vascular puncture apparatus or deviation in a position or an inclination of the vascular puncture apparatus itself occurs.

(15) A vascular puncture apparatus is disclosed comprising: a detection unit configured to detect at least one of relative positions between the vascular puncture apparatus and a puncture target, relative positions between the vascular puncture apparatus and tissue including the puncture target, a position of the vascular puncture apparatus, and an inclination of the vascular puncture apparatus; and a control unit configured to: control movement of a drive unit that includes a puncture needle; obtain a detection result before puncture from the detection unit before the puncture operation by the drive unit; continuously or intermittently obtain, after a start of the puncture operation by the drive unit, a detection result after the start of the puncture from the detection unit; calculate an amount of change in at least one of the relative positions between the vascular puncture apparatus and the puncture target, the relative positions between the vascular puncture apparatus and the tissue including the puncture target, the position of the vascular puncture apparatus, and the inclination of the vascular puncture apparatus by comparing the detection result after the start of the puncture with the detection result before the puncture; and stop the movement of the puncture needle by the drive unit or changes a movement path of the puncture needle if the at least one amount of change is larger than or equal to a threshold or larger than the threshold.

### BRIEF DESCRIPTION OF THE DRAWINGS

FIG. 1 is a perspective view of a vascular puncture apparatus according to an embodiment.

FIG. 2 is a side view of the vascular puncture apparatus according to the embodiment.

FIG. 3 is a cross-sectional view taken along line B-B in FIG. 2.

FIG. 4 is a block diagram illustrating a schematic configuration of the vascular puncture apparatus.

FIG. 5 is a side view illustrating a part of the vascular puncture apparatus and an arm.

FIG. 6 is a first half of a flowchart illustrating an operation of a control unit.

FIG. 7 is a second half of the flowchart illustrating the operation of the control unit.

FIG. 8 is a modification of part of the flowchart illustrating the operation of the control unit.

### DETAILED DESCRIPTION

Set forth below with reference to the accompanying drawings is a detailed description of embodiments of a vascular puncture apparatus that automatically punctures a blood vessel and a method for controlling the vascular puncture apparatus. Note that dimensional ratios in the drawings might be exaggerated for convenience of description and differ from actual ratios.

A vascular puncture apparatus 10 according to the embodiment of the present disclosure is used when an arm A of a human body is punctured, and obtains a cross-sectional image of the arm A, detects a position of a blood vessel as a puncture target, and automatically punctures a blood vessel V.

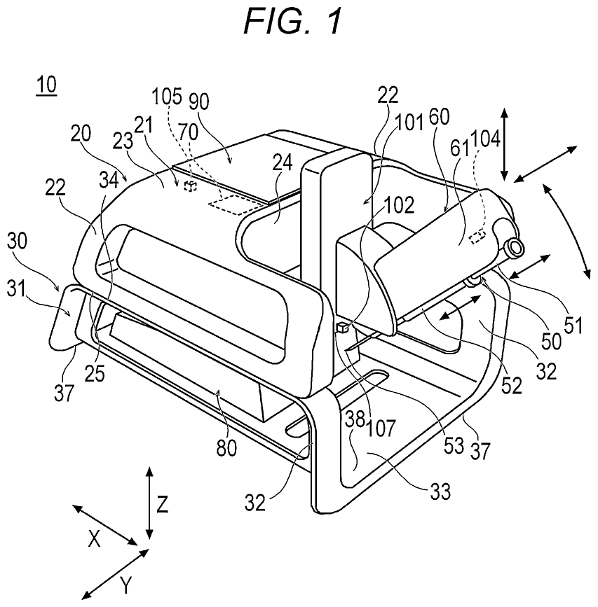

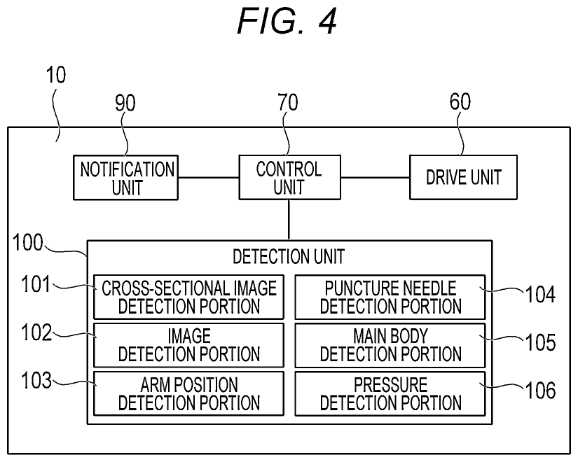

As illustrated in FIGS. 1 to 4, the vascular puncture apparatus 10 includes a main body 20 that automatically punctures the blood vessel V, a fixing base 30 that overlaps the main body 20 in a height direction Z and that is disposed under the main body 20 to support the main body 20, and a detection unit 100 that detects various states. The height direction Z is a direction perpendicular to a horizontal surface when the vascular puncture apparatus 10 is disposed on the horizontal surface. The main body 20 includes an upper frame 21, a puncture unit 50 that performs puncture, a drive unit 60 that moves the puncture unit 50 with respect to the upper frame 21, a control unit 70 that performs calculation, and a notification unit 90 capable of notifying the outside of information. The detection unit 100 includes a cross-sectional image detection portion 101 that detects a cross-sectional image of the arm A, an image detection portion 102 that detects an image of a surface (skin) of the arm A, an arm position detection portion 103 that detects a position of the arm A, a puncture needle detection portion 104 that detects the puncture unit 50, a main body detection portion 105 that detects the main body 20, and pressure detection portions 106 that detect pressure of the holding unit 80, which is disposed on the fixing base 30 and holds the arm A.

The upper frame 21 is formed in a substantially arch shape protruding upward when viewed from a length direction X corresponding to a direction in which the arm A of the human body set in the vascular puncture apparatus 10 extends. The upper frame 21 includes two upper walls 22 formed on both sides in a width direction Y orthogonal to the length direction X, a top surface portion 23 connecting the two upper walls 22 to each other, and an opening 24 in which the cross-sectional image detection portion 101, the image detection portion 102, and the puncture unit 50 are arranged. The upper frame 21 can be formed of, for example, a hard resin material.

Each upper wall 22 has a main body coupling surface 25 at a lower end of each of the upper walls 22. Each main body coupling surface 25 has at least one protrusion 26 as a coupling portion that can be coupled to the fixing base 30. In the present embodiment, each main body coupling surface 25 has one protrusion 26.

The notification unit 90 that displays an image captured by the cross-sectional image detection portion 101 is disposed on a surface of the top surface portion 23 facing upward. In the top surface portion 23, a gripping hole 27 is formed in a surface on a central side (a side close to a shoulder of the arm A) of the upper frame 21 in the length direction X toward a peripheral side (a side close to fingertips of the arm A) in the length direction X. The gripping hole 27 can be used by an operator to lift the main body 20 with a hand. The gripping hole 27 extends from the central side of the top surface portion 23 toward a center of gravity of the main body 20. Note that the gripping hole 27 may or may not reach the center of gravity of the main body 20. Since the gripping hole 27 is formed close to the center of gravity, the operator can easily lift the main body 20 with one hand without dropping the main body 20.

The opening 24 is formed in a recessed shape on the peripheral side of the upper frame 21 in the length direction X when viewed from above. That is, the top surface portion 23 is formed shorter than the upper wall 22 on the peripheral side in the length direction X, and the opening 24 is formed in the upper frame 21. The opening 24 is disposed on the peripheral side of the top surface portion 23. The arm A to be imaged by the cross-sectional image detection portion 101 and punctured by the puncture unit 50 is exposed from the opening 24. The cross-sectional image detection portion 101 is coupled to the opening 24. The cross-sectional image detection portion 101 may be removable or non-removable from the opening 24.

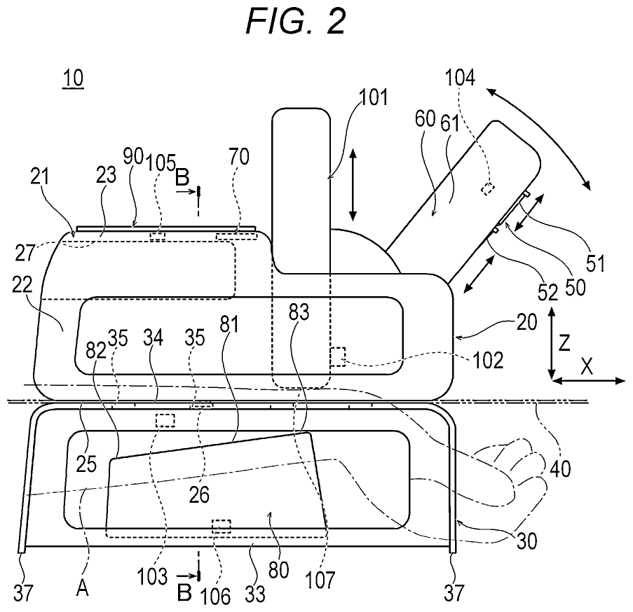

As illustrated in FIGS. 1 and 2, the puncture unit 50 includes a metal puncture needle 51 having a sharp needle tip 53 formed at a distal end of the puncture needle 51 and a flexible outer tube 52 disposed in such a way as to cover an outer peripheral surface of the puncture needle 51. In a state in which the outer tube 52 covers the outside of the puncture needle 51, the needle tip 53 of the puncture needle 51 protrudes from the outer tube 52. The puncture needle 51 and the outer tube 52 are removable from a needle holding portion 61 of the drive unit 60.

The drive unit 60 is a drive source for automatically adjusting a puncture position and performing a puncture operation by changing a position and/or an angle of the puncture unit 50 under the control of the control unit 70. The drive unit 60 is coupled to the cross-sectional image detection portion 101 or the upper frame 21 and disposed in the opening 24. The drive unit 60 includes a mechanism for tilting the entirety of the puncture unit 50 with respect to the upper frame 21 and a horizontal plane (a plane parallel to the length direction X and the width direction Y), a mechanism for moving the entirety of the puncture unit 50 in the width direction Y, a mechanism for 50 moving the puncture unit 50 in the height direction Z, a mechanism for moving the puncture needle 51 forward and backward in a puncture direction, and a mechanism for fixing the outer tube 52 to the puncture needle 51 or moving the outer tube 52 along the puncture needle 51. The drive unit 60 is configured by combining, for example, a rotary drive source, such as a motor, a structure (for example, a feed screw mechanism) that converting rotational motion of the rotary drive source into linear motion, a structure (for example, a hinge) that changes an angle, and the like, but the configuration is not limited to this. The drive unit 60 includes the needle holding portion 61 that removably holds the puncture needle 51 and the outer tube 52.

The control unit 70 obtains detection results from the cross-sectional image detection portion 101, the image detection portion 102, the arm position detection portion 103, the puncture needle detection portion 104, the main body detection portion 105, and the pressure detection portions 106, and can perform calculation for controlling operation of the detection unit 100 and the drive unit 60. The control unit 70 includes a storage circuit and an arithmetic circuit as physical components. The storage circuit can store programs and various parameters. The arithmetic circuit can perform arithmetic processing.

The control unit 70 may be disposed in the upper frame 21, or may be disposed in the cross-sectional image detection portion 101 or the drive unit 60. Alternatively, the control unit 70 may be configured separately from the main body 20.

The control unit 70 can identify a position of the blood vessel V in the cross-sectional image obtained from the cross-sectional image detection portion 101, which is an echograph, by analyzing the cross-sectional image. Furthermore, the control unit 70 can detect a relative position of the puncture needle 51 with respect to the cross-sectional image detection portion 101 by analyzing the cross-sectional image. Since the outer tube 52 is made of a resin, the outer tube 52 is not detected by ultrasonic waves. However, when the outer tube 52 is made of a resin containing metal, for example, the outer tube 52 can be detected by ultrasonic waves, and the control unit 70 can detect the relative position of the outer tube 52 with respect to the cross-sectional image detection portion 101. In addition, the control unit 70 can cause the notification unit 90 to display the cross-sectional image by analyzing the obtained cross-sectional image. Furthermore, the control unit 70 can control the drive unit 60 in such a way as to adjust the position and angle of the puncture unit 50, and automatically perform puncture using the puncture needle 51 and the outer tube 52 at a desired position and angle.

The notification unit 90 is a part that notifies the outside of information, and in the present embodiment, is a monitor capable of displaying a cross-sectional image as illustrated in FIGS. 1 to 4. Note that the notification unit 90 may be a speaker or the like that emits sound such as voice, instead. The notification unit 90 is preferably disposed near the puncture unit 50 that performs puncture. For example, the notification unit 90 is disposed on the surface of the top surface portion 23 facing upward, the surface being adjacent to the opening 24 in which the puncture unit 50 is disposed. As a result, the operator can view the skin of the arm A to be punctured while viewing a cross-sectional image of the blood vessel V on the notification unit 90. Note that notification unit 90 may be disposed away from main body 20 and fixing base 30.

As illustrated in FIGS. 1 to 3, the fixing base 30 includes a lower frame 31 and a holding unit 80 that holds the arm A. The lower frame 31 is formed in a substantially arch shape protruding downward when viewed in the length direction X. The lower frame 31 includes two lower walls 32 formed on both sides in the width direction Y and a bottom surface portion 33 connecting the two lower walls 32 to each other. The lower frame 31 can be formed of, for example, a hard resin material.

Each lower wall 32 includes a fixing base coupling surface 34 at an upper end of each of the lower walls 32. The fixing base coupling surface 34 faces the main body coupling surface 25 of the main body 20 to support the main body coupling surface 25. A drape 40 can be sandwiched between the fixing base coupling surface 34 and the main body coupling surface 25. The drape 40 is a flexible sheet-like member, and can be used to separate a sterile field and an unsterile field from each other. Configuration of the drape 40 is not particularly limited as long as the drape 40 has a flexible sheet shape. Each fixing base coupling surface 34 includes at least one recess 35 as a coupling portion that can be coupled to the main body 20. In the present embodiment, each fixing base coupling surface 34 includes a plurality of recesses 35 arranged at predetermined intervals in the length direction X. The recesses 35 may or may not penetrate the fixing base coupling surface 34. Each recess 35 can be fitted with one of protrusions 26 of the main body coupling surface 25 via the drape 40. The coupling structure between the main body 20 and the fixing base 30 is not limited to the structure in which the protrusions 26 are fitted to the recesses 35. The main body 20 and the fixing base 30, therefore, may have a structure where the coupling is achieved by magnetic force, instead. The main body 20 and the fixing base 30 include a magnet or a ferromagnetic body that has magnetic attraction. In this case, the main body 20 and the fixing base 30 need not have an uneven structure (the protrusions 26 and the recesses 35).

The bottom surface portion 33 includes a bottom surface 37 in contact with an operating table or the like and a mounting surface 38 facing upward. The holding unit 80 that holds the arm A is fixed to the mounting surface 38.

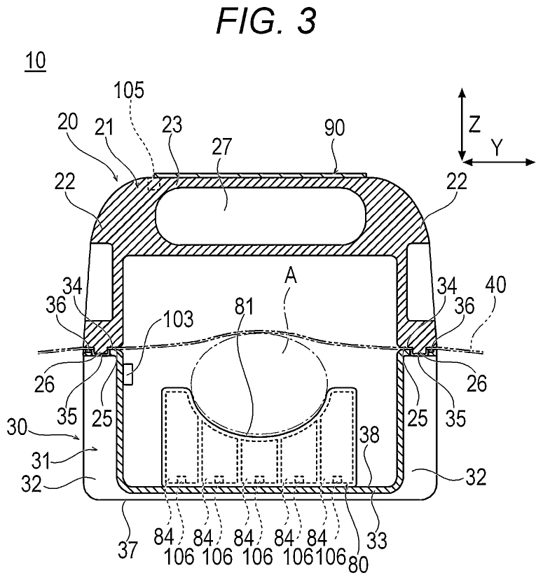

As illustrated in FIGS. 2 and 3, the holding unit 80 has a holding surface 81 capable of holding the arm A on an upper surface of holding unit 80. The holding surface 81 is formed in a groove shape in accordance with a shape of the arm A so as to stably hold the arm A in the length direction X. The holding surface 81 includes a first holding surface 82 capable of holding an elbow side (central side) of the arm A and a second holding surface 83 capable of holding a wrist side (peripheral side) of the arm A. A height from the bottom surface 37 of the fixing base 30 to the second holding surface 83 is preferably greater than a height from the bottom surface 37 to the first holding surface 82. Height of the holding surface 81 from the bottom surface 37 gradually increases from the elbow side (central side) toward the wrist side (peripheral side) in the length direction X. As a result, the arm A can be stably held by the holding unit 80 in a state in which a palm side of the wrist is turned upward to stretch the skin of the arm A (see FIG. 2). The holding unit 80 is preferably soft. As a result, since the holding unit 80 can be deformed in accordance with the shape of the arm A, the arm A can be stably held, and a pain of the patient can be reduced.

The holding unit 80 may have a soft hollow structure including hollow portions 84 that can expand or contract by allowing a fluid such as gas or liquid to flow in or out. Each hollow portion 84 can hold the arm A in a desirable state by supplying and discharging the fluid using, for example, a pump or the like whose driving is controlled by the control unit 70. The plurality of hollow portions 84 capable of accommodating the fluid may be arranged in the width direction Y, and the pressure detection portion 106 may be disposed in each of the hollow portions 84. Note that the plurality of hollow portions 84 may be arranged in the length direction X. Alternatively, only one hollow portion 84 may be provided. The holding unit 80 may be formed of, for example, a flexible porous resin material. Alternatively, the holding unit 80 need not be soft, and may have a structure integrated with the lower frame 31.

As illustrated in FIGS. 1 to 4, the cross-sectional image detection portion 101 is an echograph, and can be disposed in, for example, the upper frame 21 of the main body 20 and detects a cross-sectional image of the arm A, which is tissue including the blood vessel V as a puncture target. A horizontal direction in the cross-sectional image corresponds to the width direction Y, and a vertical direction in the cross-sectional image corresponds to the height direction Z. The cross-sectional image detection portion 101 includes, at a lower end of the cross-sectional image detection portion 101, a probe 107 that generates and transmits ultrasonic waves and that receives ultrasonic waves (echoes) reflected from the inside of a living body. The probe 107 is elongated in the width direction Y, and can obtain a cross-sectional image orthogonal to an axial direction of the blood vessel V inside the human body. Note that the cross-sectional image detection portion 101, which is an echograph, can detect feature points from shapes of the blood vessel V, muscles, tendons, bones, and the like in the cross-sectional image. Each feature point is one point determined by a shape, such as a center of gravity of a circular shape or a center of an arc shape. The cross-sectional image detection portion 101 is used to monitor the cross-sectional image and can be used to detect movement of the arm A and the blood vessel V with respect to the vascular puncture apparatus 10 and movement of the blood vessel V with respect to the arm A.

The image detection portion 102 can be disposed on, for example, the upper frame 21 of the main body 20, and is disposed downward in the height direction Z in such a way as to face the arm A held by the holding unit 80. The image detection portion 102 is a camera that images the surface of the arm A. The camera can continuously or intermittently detect an image of the surface of the arm A, and detect feature points determined from shapes of wrinkles, lentigines, veins, pores, and the like. The image detection portion 102, therefore, can detect the movement of the arm A with respect to the vascular puncture apparatus 10.

The arm position detection portion 103 is a distance sensor that is disposed on the fixing base 30 and that detects a relative distance between the arm A and the skin surface. The arm position detection portion 103 is disposed on an inner surface of one of the lower walls 32 of the fixing base 30 in such a way as to face the arm A held by the holding unit 80. The arm position detection portion 103 can detect the movement of the arm A in the width direction Y with respect to the vascular puncture apparatus 10 by facing in the width direction Y. In addition, since the arm A is roughly circular when viewed as a cross section, the arm position detection portion 103 can also detect movement of the arm A in the length direction X and movement of the arm A in the height direction Z. The number of arm position detection portions 103 may be one or more. A plurality of arm position detection portions 103, therefore, may be arranged on both inner surfaces of the lower walls 32 facing each other, or may be arranged at different positions in the length direction X. The arm position detection portion 103 is disposed on the fixing base 30 in the present embodiment, but may be disposed on the main body 20. The arm position detection portion 103 may be disposed on one or both of the inner surfaces of the upper walls 22 of the main body 20, or the plurality of arm position detection portions 103 may be arranged on the upper walls 22 at different positions in the length direction X. A type of sensor of the arm position detection portion 103 is not limited to the distance sensor as long as a distance can be estimated, and may be, for example, a sensor that detects a color tone, brightness, temperature, or the like, an image sensor, or the like, instead.

The puncture needle detection portion 104 is a proximity sensor that is disposed in the needle holding portion 61 to which the puncture needle 51 is fixed and that detects the puncture needle 51. The puncture needle detection portion 104 detects feature points on the puncture needle 51 (for example, a groove or an identification mark). The puncture needle detection portion 104 can detect whether or not the puncture needle 51 is at an appropriate position with respect to the needle holding portion 61. The number of puncture needle detection portions 104 may be one or more. A type of sensor of the puncture needle detection portion 104 is not limited to the proximity sensor as long as feature points on the puncture needle 51 can be detected, and may be, for example, a distance sensor, a sensor that detects a color tone, brightness, or the like, an image sensor, or the like, instead.

The main body detection portion 105 is an inclination sensor (gyro sensor) and/or an inertial sensor (acceleration sensor) that is disposed in the main body 20 and that detects an inclination and/or a position of the main body 20. The main body detection portion 105 can detect whether or not the main body 20 is at an appropriate inclination and/or position. The number of main body detection portions 105 may be one or more. The main body detection portion 105 may be disposed on the fixing base 30. The main body detection portion 105, therefore, detects an inclination and/or a position of the vascular puncture apparatus 10 (the main body 20 and/or the fixing base 30). The position of the vascular puncture apparatus 10 is spatial coordinates of the vascular puncture apparatus 10.

As described above, the pressure detection portion 106 is disposed in each of the plurality of hollow portions 84 of the holding unit 80. By arranging the pressure detection portion 106 in each hollow portion 84, it is possible to detect a direction in which the holding unit 80 is receiving force from the arm A. The pressure detection portions 106 can continuously detect pressure and detect the displacement of the arm A on the basis of changes in the pressure. The pressure detection portions 106, therefore, can also function as the arm position detection portions 103.

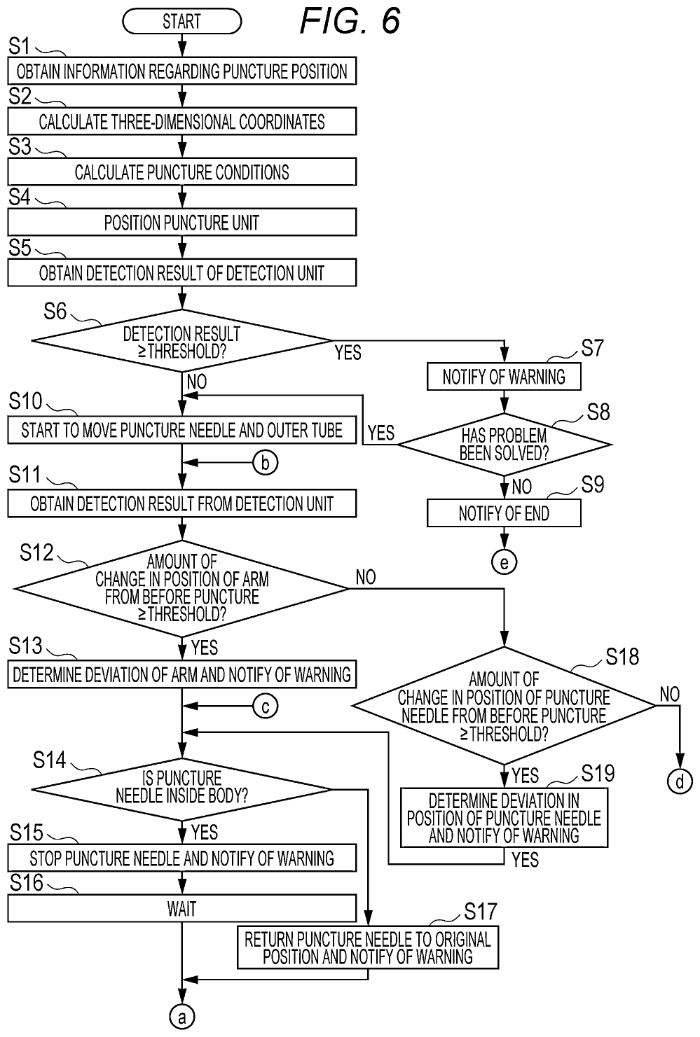

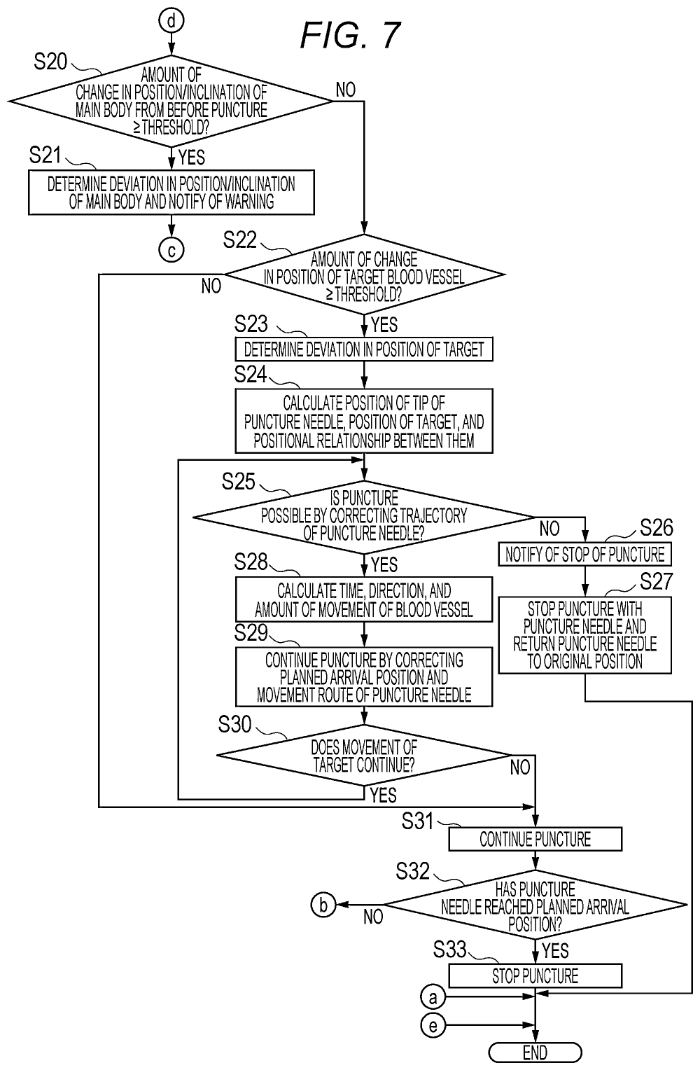

Next, a method for puncturing the blood vessel V as a puncture target using the vascular puncture apparatus 10 according to the present embodiment will be described with reference to flowcharts of FIGS. 6 and 7 used by the control unit 70.

First, the operator places the fixing base 30 on an operating table or the like, and sets the arm A of the patient on the holding surface 81. Next, as illustrated in FIGS. 2 and 3, the operator fits a hole in the drape 40 to a site of the arm A where puncture is to be performed, and covers the fixing base 30 and the arm A with the drape 40. Next, the operator disposes the main body 20 on the fixing base 30 while sandwiching the drape 40, and fits the protrusions of the main body 20 to the recesses 35 in the fixing base 30.

Next, the operator actuates the vascular puncture apparatus 10. The control unit 70 obtains information regarding a puncture position S determined by another program (step S1). Calculation of the other program may be performed by the control unit 70 or may be performed by another device. When the puncture position is determined by another device, the control unit 70 is connected to the other device and obtains information from the other device.

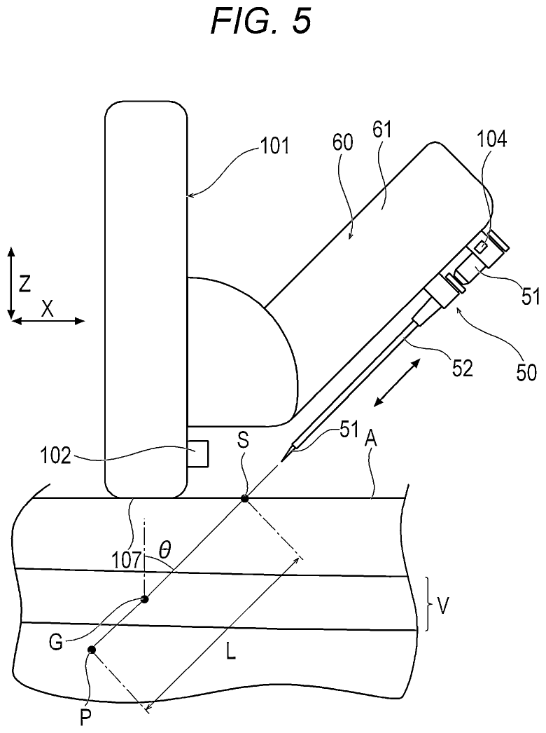

Next, as illustrated in FIG. 5, the control unit 70 calculates three-dimensional coordinates of the puncture position S on the skin surface, a center of gravity G through which the puncture needle 51 for the blood vessel V passes, and a planned arrival position P (step S2). The planned arrival position P is the deepest position where the tip of the puncture needle 51 is planned to arrive. In order to identify the position of the blood vessel V in the cross-sectional image, the control unit 70 can prepare a large number of images of the same type and use a method of machine learning or deep learning. In addition, it is also possible to detect a region with blood flow by a Doppler method in the probe 107 and recognize the region as a region of the blood vessel V.

In addition, the control unit 70 calculates puncture conditions such as a puncture angle θ, a target puncture depth L, and a puncture speed (step S3). The puncture angle θ is an angle at which the puncture needle 51 at a time of puncture is inclined with respect to a perpendicular line of the skin surface. The puncture angle θ may be, for example, a preset angle (for example, 30 degrees), instead. The target puncture depth L is a distance from the puncture position S on the skin surface to the planned arrival position P after passing through the center of gravity G of the blood vessel V. Note that the planned arrival position P may be changed through calculation by the control unit 70 in accordance with a situation at the time of puncture.

Next, the control unit 70 controls the drive unit 60 such that the puncture needle 51 can perform puncture while satisfying the puncture position S and the puncture angle θ, and positions the puncture unit 50 at a desired position (coordinates) in a desired posture (angle) (step S4).

Next, before starting a puncture operation, the control unit 70 obtains a detection result from each sensor of the detection unit (step S5). Specifically, the control unit 70 obtains a detection result from at least one of the cross-sectional image detection portion 101, the image detection portion 102, the arm position detection portion 103, the puncture needle detection portion 104, the main body detection portion 105, and the pressure detection portion 106. The control unit 70 can calculate, from the detection results of the cross-sectional image detection portion 101, the image detection portion 102, and the puncture needle detection portion 104, the above-described feature points through calculation set for these portions, and use the feature points as detection results. The control unit 70 compares each of the obtained detection results with a threshold set in advance, and determines whether or not the detection result is larger than or equal to the threshold (or larger than the threshold) (step S6).

If determining in step S6 that the obtained detection result is larger than the threshold (or larger than or equal to the threshold), the control unit 70 causes the notification unit 90 to notify of information indicating a warning together with information for identifying a sensor in which a problem has occurred (step S7). As a result, the operator can identify the sensor in which the problem has occurred and solve the problem. After step S7, the control unit 70 determines whether or not the problem has been solved (step S8), and if the problem has been solved, proceeds to step S10, in which the puncture operation is started. If determining in step S8 that the problem has not been solved, the control unit 70 causes the notification unit 90 to notify of information indicating an end of automatic puncture (step S9), and ends the automatic puncture.

If determining in step S6 that the obtained detection result is smaller than the threshold (or smaller than or equal to the threshold) and determining in step S8 that the problem has been solved, the control unit 70 controls the drive unit 60 in such a way as to start integral movement of the puncture needle 51 and the outer tube 52 (step S10). The control unit 70 continuously or intermittently obtains a detection result from each sensor of the detection unit 100 during the puncture operation by the drive unit 60 (step S11).

The control unit 70 determines whether or not the amount of change in the position of the arm A detected during the puncture with respect to the position before the puncture is larger than or equal to a threshold (or larger than the threshold) (step S12). The amount of change in the position of the arm A can be calculated by at least one of a plurality of methods described below. A first example of the amount of change in the position of the arm A with respect to the position before the puncture is the amount of change in a position of a feature point in the cross-sectional image obtained from the cross-sectional image detection portion 101, which is an echograph, during the puncture with respect to a value before the puncture. A second example of the amount of change in the position of the arm A with respect to the position before the puncture is the amount of change in a position of a feature point in the image obtained from the image detection portion 102, which is a camera, during the puncture with respect to a value before the puncture. A third example of the amount of change in the position of the arm A with respect to the position before the puncture is the amount of change in a value obtained from the arm position detection portion 103, which is a distance sensor, during the puncture with respect to a value before the puncture. A fourth example of the amount of change in the position of the arm A with respect to the position before the puncture is the amount of change in a value obtained from the pressure detection portion 106 including a pressure sensor during the puncture with respect to a value before the puncture.

If determining in step S12 that the obtained detection result is larger than or equal to the threshold (or larger than the threshold), the control unit 70 causes the notification unit 90 to notify of a warning indicating that deviation in the position of the arm A has occurred (step S13). Note that deviation in the position of the arm A can occur when the patient consciously or unconsciously moves the arm A due to discomfort, pain, surprise, or the like before or after the puncture needle 51 passes through the skin. Next, the control unit 70 determines whether or not the tip of the puncture needle 51 is in the body (step S14). The determination whether or not the puncture needle 51 is in the body can be calculated by at least one of a plurality of methods described below. A first example of the determination whether or not the puncture needle 51 is in the body is a method in which a position of the tip of the puncture needle 51 is checked using a cross-sectional image obtained by the cross-sectional image detection portion 101, which is an echograph. A second example of the determination whether or not the puncture needle 51 is in the body is a method for making the determination on the basis of a relationship between the position of the tip of the puncture needle 51 calculated from the puncture angle θ and a distance of forward or backward movement of the puncture needle 51 and a position of the probe 107 (a position of the skin surface). A third example of the determination whether or not the puncture needle 51 is in the body is a method for making the determination on the basis of a measurement result of a force sensor that detects a force acting on the puncture needle 51.

If determining in step S14 that the tip of the puncture needle 51 is in the body, the control unit 70 controls the drive unit 60 in such a way as to stop the movement of the puncture needle 51 and the outer tube 52, and causes the notification unit 90 to notify of a warning indicating that the puncture needle 51 has been stopped due to the occurrence of the deviation in the position of the arm A (step S15). Next, the control unit 70 enters a standby state until there is an operation by the operator (step S16), and ends the control.

If determining in step S14 that the tip of the puncture needle 51 is not in the body, the control unit 70 controls the drive unit 60 in such a way as to stop the movement of the puncture needle 51 and the outer tube 52 and return the puncture needle 51 and the outer tube 52 to original positions, causes the notification unit 90 to notify of information for requesting re-fixation of the puncture needle 51 and the main body 20 (step S17), and ends the control.

If determining in step S12 that the obtained detection result is smaller than the threshold (or smaller than or equal to the threshold), the control unit 70 determines that deviation in the position of the arm A has not occurred. Subsequently, the control unit 70 determines whether or not the amount of change in a detection result of the puncture needle detection portion 104 detected after the start of the puncture operation with respect to a detection result before the start of the puncture operation is larger than or equal to a threshold (or larger than the threshold) (step S18). That is, the control unit 70 determines whether or not deviation in the position of the puncture needle 51 with respect to the needle holding portion 61 has occurred. If determining in step S18 that the amount of change in the detection result of the puncture needle detection portion 104 is larger than or equal to the threshold (or larger than the threshold), the control unit 70 causes the notification unit 90 to notify of a warning indicating that deviation in the position of the puncture needle 51 with respect to the needle holding portion 61 has occurred (step S19). Next, the control unit 70 proceeds to the determination in step S14 described above whether or not the tip of the puncture needle 51 is in the body. Note that deviation in the position of the puncture needle 51 with respect to the needle holding portion 61 can occur due to an error in a fixing position, incomplete fixing of the puncture needle 51, or movement of the vascular puncture apparatus 10 due to shaking of the table on which the vascular puncture apparatus 10 is placed before or after the puncture needle 51 passes through the skin.

If determining in step S18 that the amount of change in the detection result of the puncture needle detection portion 104 is smaller than the threshold (or smaller than or equal to the threshold), the control unit 70 determines that deviation in the position of the puncture needle 51 with respect to the needle holding portion 61 has not occurred. Subsequently, the control unit 70 determines whether or not the amount of change in a detection result of the main body detection portion 105 detected after the start of the puncture operation with respect to a detection result before the start of the puncture operation is larger than or equal to a threshold (or larger than the threshold) (step S20). That is, whether a position and/or an inclination of the main body 20 is deviated is determined. If determining in step S20 that the amount of change in the detection result of the main body detection portion 105 is larger than or equal to the threshold (or larger than the threshold), the control unit 70 causes the notification unit 90 to notify of a warning indicating that the position and/or inclination of the main body 20 is deviated (step S21). Next, the control unit 70 proceeds to the determination in step S14 described above whether or not the tip of the puncture needle 51 is in the body.

If determining in step S20 that the amount of change in the detection result of the main body detection portion 105 is smaller than the threshold (or smaller than or equal to the threshold), the control unit 70 determines that deviation in the position or inclination of the main body 20 has not occurred. Subsequently, the control unit 70 determines whether or not the amount of change in a position of the blood vessel V detected after the start of the puncture operation with respect to a detection result before the start of the puncture operation is larger than or equal to a threshold (or larger than the threshold) (step S22). That is, it is determined whether or not only the position of the blood vessel V as the puncture target has changed whereas deviation of the positions of the arm A, the puncture needle 51, and the main body 20 has not occurred. A change in a target position can be identified from the amount of change in a position of a contour of the blood vessel V as the puncture target identified from the cross-sectional image obtained from the cross-sectional image detection portion 101, which is an echo graph, from a position before the puncture. Since the control unit 70 makes the determination in step S22 after determining in step S12, step S18, and step S20 that deviation of the arm A, the puncture needle 51, and the main body 20 has not occurred, the control unit 70 can determine that only the blood vessel V as the puncture target is moving. If determining in step S22 that the amount of change in the position of the blood vessel V as the puncture target is smaller than the threshold (or smaller than or equal to the threshold), the control unit 70 continues the puncture and returns to step S11 to repeat the detection by each sensor and each determination.

If determining in step S22 that the amount of change in the position of the blood vessel V is larger than or equal to the threshold (or larger than the threshold), the control unit 70 determines that deviation in the position of the blood vessel V as the puncture target has occurred (step S23), and attempts to correct a movement path of the puncture needle 51. For this purpose, the control unit 70 calculates the position of the tip of the puncture needle 51 and the position of the blood vessel V as the puncture target, and calculates a positional relationship between the two (step S24). Next, the control unit 70 determines whether or not puncture of the blood vessel V as the puncture target is possible by correcting the movement path of the tip of the puncture needle 51 (step S25).

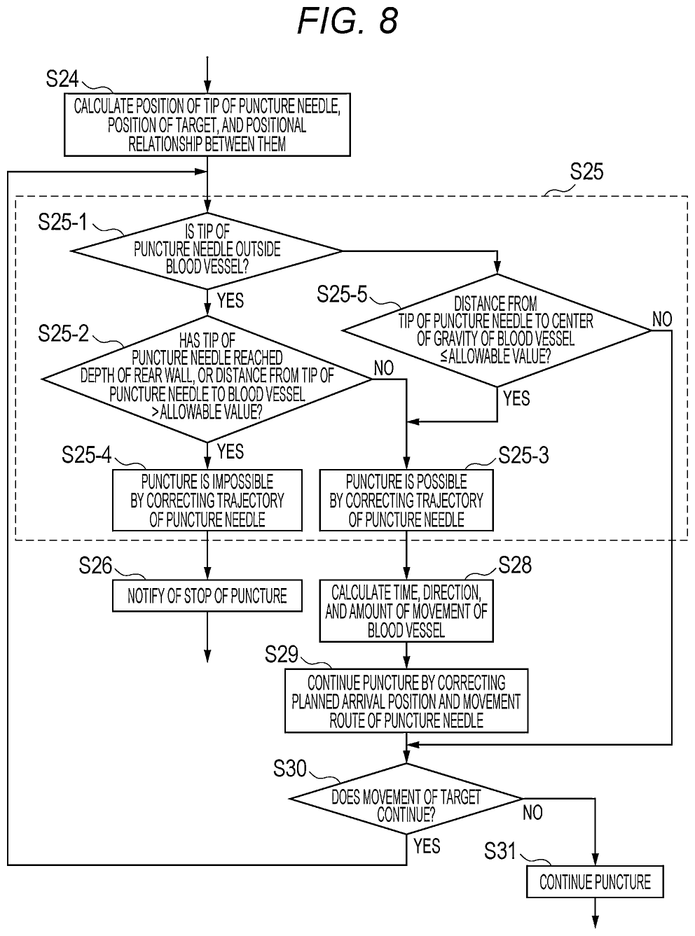

FIG. 8 illustrates a specific modification of step S25. After the puncture needle 51 reaches a calculated puncture depth, the control unit 70 determines whether the position of the tip of the puncture needle 51 is located inside or outside the blood vessel V in the cross-sectional image (step S25-1). If the position of the tip of the puncture needle 51 is located outside the blood vessel V, the control unit 70 determines whether the position of the tip of the puncture needle 51 has reached the same depth as a rear wall of the blood vessel V, or whether a distance from the position of the tip of the puncture needle 51 to the center of gravity G (or a vessel wall) of the blood vessel V identified from the cross-sectional image exceeds a preset allowable value (step S25-2). Among vessel walls, a wall of the blood vessel V on a side close to the skin to be punctured is a front wall, and a wall of the blood vessel V on a side away from the skin to be punctured is a rear wall. The control unit 70 can identify positions of the vessel walls, namely the front wall and the rear wall, from position information regarding the blood vessel V. If determining in step S25-2 that the position of the tip of the puncture needle 51 has not reached the same depth as the rear wall of the blood vessel V and the distance from the position of the tip of the puncture needle 51 to the center of gravity G (or the vessel wall) of the blood vessel V is within the preset allowable value, the control unit 70 can determine that the puncture of the blood vessel V is possible by correcting the movement path of the tip of the puncture needle 51 (step S25-3). The control unit 70 then calculates the movement path from the position of the tip of the puncture needle 51 to the planned arrival position P again, and moves the puncture needle 51 toward the identified blood vessel V by the calculated distance (step S28 and step S29). Note that the planned arrival position P may be an initially determined position, or may be a position calculated again.

If determining in step S25-2 that the position of the tip of the puncture needle 51 has reached the same depth as the rear wall of the blood vessel V or that the distance from the position of the tip of the puncture needle 51 to the center of gravity G (or the vessel wall) of the blood vessel V exceeds the preset allowable value, the control unit 70 can determine that the puncture of the blood vessel V is impossible by correcting the movement path of the tip of the puncture needle 51 (step S25-4).

If determining in step S25-1 that the position of the tip of the puncture needle 51 is located inside the blood vessel V, the control unit 70 calculates the distance from the position of the tip of the puncture needle 51 to the center of gravity G of the blood vessel V identified from the cross-sectional image, and determines whether or not the distance is within the preset allowable value (step S25-5). If determining that the distance from the position of the tip of the puncture needle 51 to the center of gravity G exceeds the allowable value, the control unit 70 can determine that the puncture of the blood vessel V as the puncture target is possible by correcting the movement path of the tip of the puncture needle 51 (step S25-3). If determining that the distance from the position of the tip of the puncture needle 51 to the center of gravity G is smaller than or equal to the allowable value, the control unit 70 determines that trajectory correction of the puncture needle 51 is unnecessary, and proceeds to step S30.

Note that the determination by the control unit 70 in step S25 whether or not the blood vessel V as the puncture target can be punctured by correcting the movement path of the tip of the puncture needle 51 is not limited to the determination based on whether or not the position of the tip of the puncture needle 51 has reached the same depth as the rear wall. For example, an allowable value of deviation in the position of the tip of the puncture needle 51 in the Y-coordinate direction with respect to the position of the blood vessel V may be set for the control unit 70. If the deviation in the position of the puncture needle 51 in the Y-coordinate direction with respect to the position of the blood vessel V is smaller than or equal to the allowable value, the control unit 70 can determine that the puncture of the blood vessel V is possible by correcting the movement path of the tip of the puncture needle 51. If the deviation in the position of the puncture needle 51 in the Y-coordinate direction with respect to the position of the blood vessel V exceeds the allowable value, the control unit 70 can determine that the puncture of the blood vessel V as the puncture target is impossible by correcting the movement path of the tip of the puncture needle 51. As illustrated in FIG. 7, if determining in step S25 that the puncture of the blood vessel V is impossible by correcting the movement path of the tip of the puncture needle 51, the control unit 70 causes the notification unit 90 to notify of a warning indicating that the position of the blood vessel V as the puncture target has changed (step S26). Subsequently, the control unit 70 controls the drive unit 60 in such a way as to stop the movement of the puncture needle 51 and the outer tube 52 and return the puncture needle 51 and the outer tube 52 to original positions (step S27). Note that changes in the position and diameter of the blood vessel V can be caused by vasospasm and movement of muscles. Furthermore, at the time of puncture, changes in the position and diameter of the blood vessel V can also be caused when perivascular tissue is deformed by the movement of the puncture needle 51 and the blood vessel V is pushed.

If determining in step S25 that the puncture of the blood vessel V as the puncture target is possible by correcting the movement path of the tip of the puncture needle 51, the control unit 70 calculates movement time, a movement direction, and the amount of movement of the blood vessel V from a difference between the position of the blood vessel V before the puncture and a current position of the blood vessel V (step S28). Next, the control unit 70 corrects the planned arrival position P and the movement path of the tip of the puncture needle 51 in accordance with the amount of movement, and continues the puncture operation by the drive unit 60 (step S29).

Next, the control unit 70 determines whether or not the movement of the blood vessel V as the puncture target continues (step S30). If determining that the movement of the blood vessel V as the puncture target continues, the control unit 70 returns to step S25, and can make the determination as to the correction of the movement path or recalculate the movement path or the like in accordance with the moved blood vessel V. At this time, the control unit 70 may pull back the puncture needle 51 to the position of the puncture needle 51 at a time when the blood vessel V started to move. In particular, if the tip of the puncture needle 51 affects the position of the blood vessel V (if the blood vessel V moves due to the movement of the puncture needle 51), it is effective to pull back the puncture needle 51 once and then correct the movement path.

If determining in step S30 that the movement of the blood vessel V of the puncture target does not continue, the control unit 70 continues the puncture operation by the drive unit 60 (step S31). Next, the control unit 70 determines whether the tip of the puncture needle 51 has reached the planned arrival position P from control information regarding the drive unit 60 and a latest cross-sectional image obtained from the cross-sectional image detection portion 101 (step S32). If determining in step S32 that the tip of the puncture needle 51 has reached the planned arrival position P, the control unit 70 determines that the puncture has been completed, stops the driving of the puncture needle 51 and the outer tube 52 by the drive unit 60, and stops the puncture (step S33). As a result, the puncture with the puncture needle 51 is normally completed.

If determining in step S32 that the tip of the puncture needle 51 has not reached the planned arrival position P, the control unit 70 returns to step S11 and continues the puncture.

If determining in step S22 that the amount of change in the position of the blood vessel V as the puncture target is smaller than the threshold (or smaller than or equal to the threshold), the control unit 70 determines that deviation in the position of the blood vessel V as the puncture target has not occurred, and proceeds to step S31 to continue the puncture.

When the vascular puncture by the vascular puncture apparatus 10 is completed, the operator removes the puncture needle 51 and the outer tube 52 from the needle holding portion 61. Next, the operator grips the gripping hole 27 of the main body 20 and lifts the main body 20 to detach and remove the main body 20 from the fixing base 30. After removing the main body 20 from the fixing base 30, the operator removes the puncture needle 51 while leaving the outer tube 52. As a result, the operator can perform a catheter technique via the outer tube 52.

As described above, the vascular puncture apparatus 10 according to the present embodiment is the vascular puncture apparatus 10 including the fixing base 30 including the holding unit 80 capable of holding a part of the human body, the notification unit 90 that notifies of information using an image and/or sound, the drive unit 60 that moves the puncture needle 51, the control unit 70 capable of controlling the movement of the drive unit 60, and the detection unit 100 that detects at least one of the relative positions between the vascular puncture apparatus 10 and the puncture target (blood vessel V), the relative positions between the vascular puncture apparatus 10 and the tissue (arm A) including the puncture target, the position of the vascular puncture apparatus 10, and the inclination of the vascular puncture apparatus 10, in which the control unit 70 obtains the detection result before the puncture from the detection unit 100 before the puncture operation by the drive unit 60, the control unit 70 continuously or intermittently obtains, after the start of the puncture operation by the drive unit 60, the detection result after the start of the puncture from the detection unit 100, the control unit 70 calculates the amount of change in at least one of the relative positions between the vascular puncture apparatus 10 and the puncture target (blood vessel V), the relative positions between the vascular puncture apparatus 10 and the tissue (arm A) including the puncture target, the position of the vascular puncture apparatus 10, and the inclination of the vascular puncture apparatus 10 by comparing the detection result after the start of the puncture with the detection result before the puncture, and the control unit 70 stops the movement of the puncture needle 51 by the drive unit 60 or changes a movement path of the puncture needle 51 if the at least one amount of change is larger than or equal to a threshold or larger than the threshold. As a result, the vascular puncture apparatus 10 can automatically perform or stop appropriate puncture even when deviation in the position of the puncture target or the tissue including the puncture target with respect to the vascular puncture apparatus 10 or deviation in a position or an inclination of the vascular puncture apparatus 10 itself occurs.

The detection unit 100 includes the cross-sectional image detection portion 101, which is an echograph that obtains a cross-sectional image, and the control unit 70 detects a feature point indicating a specific site from the cross-sectional image and calculates the position of the puncture target or the tissue including the puncture target. As a result, the vascular puncture apparatus 10 can detect deviation in the position of the puncture target or the tissue including the puncture target with respect to the vascular puncture apparatus 10 from the cross-sectional image obtained from the cross-sectional image detection portion 101.

The feature point is at least one of a shape of a blood vessel, a contour of a bone, or a contour of a muscle. As a result, the vascular puncture apparatus 10 can easily detect at least one of the shape of the blood vessel, the contour of the bone, and the contour of the muscle from the cross-sectional image, and calculate the position of the puncture target (blood vessel V) or the tissue (arm A) including the puncture target.

The detection unit 100 includes the image detection portion 102, which is a camera that obtains an image, and the control unit 70 detects a feature point indicating a specific site from the image and calculates the position of the tissue including the puncture target. As a result, the vascular puncture apparatus 10 can detect deviation in the position of the puncture target or the tissue including the puncture target with respect to the vascular puncture apparatus 10 from the image obtained from the image detection portion 102.

The feature point is at least one of a shape of an arm, a position of a vein, size of a lentigo, or a position of a lentigo. As a result, the vascular puncture apparatus 10 can easily detect at least one of the shape of the arm, the position of the vein, the size of the lentigo, and the position of the lentigo from the image as a feature point and calculate the position of the tissue (arm A) including the puncture target.

The detection unit 100 includes the arm position detection portion 103 that detects a relative distance from the vascular puncture apparatus 10 to an object held by the holding unit 80, and the control unit 70 calculates the position of the tissue including the puncture target from the relative distance obtained from the arm position detection portion 103. As a result, the vascular puncture apparatus 10 can detect deviation in the position of the tissue including the puncture target with respect to the vascular puncture apparatus 10 from the relative distance obtained from the arm position detection portion 103.

The drive unit 60 includes the needle holding portion 61 that fixes the puncture needle 51, the detection unit 100 includes the puncture needle detection portion 104 that is disposed in the needle holding portion 61 and that detects the puncture needle 51, and the control unit 70 calculates the position of the puncture needle 51 from the detection result obtained from the puncture needle detection portion 104. As a result, the vascular puncture apparatus 10 can detect deviation in the position of the puncture needle 51 with respect to the needle holding portion 61 from the detection result obtained from the puncture needle detection portion 104.

The detection unit 100 includes the main body detection portion 105 that is disposed in the vascular puncture apparatus 10 and that detects an absolute position or an inclination. As a result, the vascular puncture apparatus 10 can detect deviation in the position and inclination of the vascular puncture apparatus 10 from the detection result obtained from the main body detection portion 105.

The holding unit 80 includes the plurality of pressure sensors, the detection unit 100 includes the pressure detection portion 106 including the plurality of pressure sensors arranged in the holding unit 80, and the control unit 70 calculates the position of the tissue including the puncture target from the detection result obtained from the pressure detection portion 106. As a result, the vascular puncture apparatus 10 can detect deviation in the position of an arm, which is the tissue including the puncture target, with respect to the holding unit 80 from the detection result obtained from the pressure detection portion 106.

The control unit 70 determines, if the amount of change in the position of the tissue including the puncture target is smaller than or equal to the threshold or smaller than the threshold and the amount of change in the position of the puncture target is larger than or equal to the threshold or larger than the threshold, whether or not puncture of the puncture target is possible by controlling the drive unit 60 and changing the movement path of the puncture needle 51 from information regarding the position of the tip of the puncture needle 51 and the position of the puncture target, changes the movement path of the puncture needle 51 if determining that the puncture of the puncture target is possible, and stops the movement of the puncture needle 51 if determining that the puncture of the puncture target is impossible. As a result, the vascular puncture apparatus 10 can determine that only the position of the blood vessel V as the puncture target has moved whereas the arm has not moved with respect to the vascular puncture apparatus 10, change the movement path of the puncture needle 51 if the puncture of the blood vessel V is possible by changing the movement, and stop the puncture of the puncture needle 51 if the puncture of the blood vessel V is impossible by changing the movement path of the puncture needle 51.

The control unit 70 determines, if the amount of change in the position of the tissue including the puncture target is smaller than the threshold or smaller than or equal to the threshold and the amount of change in the position of the puncture target is larger than or equal to the threshold or larger than the threshold, whether or not the position of the tip of the puncture needle 51 has reached the same depth as the rear wall of the blood vessel V from the information regarding the position of the tip of the puncture needle 51 and the position of the puncture target, changes the movement path of the puncture needle 51 if determining that the position of the tip of the puncture needle 51 has not reached the same depth as the rear wall of the blood vessel V, and stops the movement of the puncture needle 51 if determining that the position of the tip of the puncture needle has reached the same depth as the rear wall of the blood vessel V. As a result, if determining that only the position of the blood vessel V as the puncture target has moved whereas the arm has not moved with respect to the vascular puncture apparatus 10, the vascular puncture apparatus 10 can change the movement path while assuming that the puncture of the blood vessel V is possible by changing the movement path of the puncture needle 51 if the position of the tip of the puncture needle 51 has not reached the same depth as the rear wall of the blood vessel V, and stop the puncture with the puncture needle 51 while assuming that the puncture of the blood vessel V is impossible by changing the movement path of the puncture needle 51 if the position of the tip of the puncture needle 51 has reached the same depth as the rear wall of the blood vessel V. This change in the movement path makes it easier to puncture the blood vessel V when the blood vessel V deviates in the Y-coordinate direction.

The control unit 70 determines, if the amount of change is larger than or equal to the threshold or larger than the threshold, whether or not the tip of the puncture needle 51 is in the body, and the control unit 70 stops the movement of the puncture needle 51 by the drive unit 60 if determining that the tip of the puncture needle 51 is in the body, and the control unit 70 stops, if determining that the tip of the puncture needle 51 is not in the body, the movement of the puncture needle 51 by the drive unit 60 and returns the puncture needle 51 to an original position. As a result, if the tip of the puncture needle 51 is in the body, the vascular puncture apparatus 10 can stop the puncture needle 51 in order to wait for a determination by the operator. Furthermore, if the tip of the puncture needle 51 is outside the body, the vascular puncture apparatus 10 can suppress erroneous puncture by returning the puncture needle 51 to the original position, and improve workability thereafter.

The control unit 70 calculates the amount of change in the position of the puncture target or the tissue including the puncture target or the amount of change in the position and/or the inclination of the vascular puncture apparatus 10 itself by comparing detection results obtained by the detection unit 100 (at least one of the cross-sectional image detection portion 101, the image detection portion 102, the arm position detection portion 103, the puncture needle detection portion 104, the main body detection portion 105, and the pressure detection portion 106) at any two points in time after the start of the puncture operation by the drive unit 60, and the control unit 70 stops the movement of the puncture needle 51 by the drive unit 60 or changes the movement path of the puncture needle 51 if the amount of change is larger than or equal to the threshold or larger than the threshold. As a result, the vascular puncture apparatus 10 can detect deviation in the position of the puncture target or the tissue including the puncture target and deviation in the position of the vascular puncture apparatus 10, which occur after the start of the puncture operation, and automatically perform or stop appropriate puncture.

A method for controlling the vascular puncture apparatus 10 according to the present embodiment is a method for controlling the vascular puncture apparatus 10 including the fixing base 30 including the holding unit 80 capable of holding a part of the human body, the notification unit 90 that notifies of information using an image and/or sound, the drive unit 60 that moves the puncture needle 51, and the control unit 70 capable of controlling the movement of the drive unit 60, the method including the steps of obtaining, before the puncture operation by the drive unit 60, the detection result before the puncture using the detection unit 100 capable of detecting at least one of the relative positions between the vascular puncture apparatus 10 and the puncture target, the relative positions between the vascular puncture apparatus 10 and the tissue including the puncture target, the position of the vascular puncture apparatus 10, and the inclination of the vascular puncture apparatus 10, continuously or intermittently obtaining, after the start of the puncture operation by the drive unit 60, the detection result after the start of the puncture from the detection unit 100, calculating the amount of change in at least one of the relative positions between the vascular puncture apparatus 10 and the puncture target, the relative positions between the vascular puncture apparatus 10 and the tissue including the puncture target, the position of the vascular puncture apparatus 10, and the inclination of the vascular puncture apparatus 10 by comparing the detection result after the start of the puncture with the detection result before the puncture, and stopping the movement of the puncture needle 51 by the drive unit 60 or changes the movement path of the puncture needle 51 if the at least one amount of change is larger than or equal to a threshold or larger than the threshold. As a result, the method for controlling the vascular puncture apparatus 10 can automatically perform or stop appropriate puncture even when deviation in the position of the puncture target or the tissue including the puncture target with respect to the vascular puncture apparatus 10 or deviation in a position or an inclination of the vascular puncture apparatus 10 itself occurs.

Note that the present disclosure is not limited to the embodiment described above, and those skilled in the art can make various modifications within the technical idea of the present disclosure. For example, in the present embodiment, the puncture is performed by a method in which the puncture needle 51 is stuck into both the front wall and the rear wall of the blood vessel V and then retracted and pulled out from the rear wall, that is, so-called double-wall puncture (DWP). The puncture may be performed by a method in which only the front wall of the blood vessel V is stuck with the puncture needle 51, that is, so-called single-wall puncture (SWP), instead.

The detailed description above describes embodiments of a vascular puncture apparatus that automatically punctures a blood vessel and a method for controlling the vascular puncture apparatus. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A vascular puncture apparatus comprising:
a fixing base including a holding unit configured to hold a part of a human body;
a notification unit configured to notify of information using an image and/or sound;
a drive unit configured to move a puncture needle;
a control unit configured to control the movement of the drive unit;
a detection unit configured to detect at least one of relative positions between the vascular puncture apparatus and a puncture target, relative positions between the vascular puncture apparatus and tissue including the puncture target, a position of the vascular puncture apparatus, and an inclination of the vascular puncture apparatus; and
the control unit is configured to:
obtain a detection result before puncture from the detection unit before the puncture operation by the drive unit;
continuously or intermittently obtain, after a start of the puncture operation by the drive unit, a detection result after the start of the puncture from the detection unit,
calculate an amount of change in at least one of the relative positions between the vascular puncture apparatus and the puncture target, the relative positions between the vascular puncture apparatus and the tissue including the puncture target, the position of the vascular puncture apparatus, and the inclination of the vascular puncture apparatus by comparing the detection result after the start of the puncture with the detection result before the puncture; and
stop the movement of the puncture needle by the drive unit or changes a movement path of the puncture needle if the at least one amount of change is larger than or equal to a threshold or larger than the threshold.

2. The vascular puncture apparatus according to claim 1, wherein
the detection unit includes a cross-sectional image detection portion, which is an echograph that obtains a cross-sectional image; and
the control unit is configured to detect a feature point indicating a specific site from the cross-sectional image and calculate the position of the puncture target or the tissue including the puncture target.

3. The vascular puncture apparatus according to claim 2, wherein the feature point is at least one of a shape of a blood vessel, a contour of a bone, or a contour of a muscle.

4. The vascular puncture apparatus according to claim 1, wherein
the detection unit includes an image detection portion, which is a camera that obtains an image; and
the control unit is configured to detect a feature point indicating a specific site from the image and calculate the position of the tissue including the puncture target.

5. The vascular puncture apparatus according to claim 4, wherein the feature point is at least one of a shape of an arm, a position of a vein, size of a lentigo, or a position of a lentigo.

6. The vascular puncture apparatus according to claim 1, wherein
the detection unit includes an arm position detection portion that detects a relative distance from the vascular puncture apparatus to an object held by the holding unit; and
the control unit is configured to calculate the position of the tissue including the puncture target from the relative distance obtained from the arm position detection portion.

7. The vascular puncture apparatus according to claim 1, wherein
the drive unit includes a needle holding portion that fixes the puncture needle;
the detection unit includes a puncture needle detection portion that is disposed in the needle holding portion and that detects the puncture needle; and
the control unit is configured to calculate a position of the puncture needle from a detection result obtained from the puncture needle detection portion.

8. The vascular puncture apparatus according to claim 1, wherein
the detection unit includes a main body detection portion that is disposed in the vascular puncture apparatus and that detects an absolute position or an inclination.

9. The vascular puncture apparatus according to claim 1, wherein
the holding unit includes a plurality of pressure sensors;
the detection unit includes a pressure detection portion including the plurality of pressure sensors arranged in the holding unit; and
the control unit is configured to calculate a position of the tissue including the puncture target from a detection result obtained from the pressure detection portion.

10. The vascular puncture apparatus according to claim 1, wherein the control unit is configured to:
determine, if the amount of change in the position of the tissue including the puncture target is smaller than or equal to the threshold or smaller than the threshold and the amount of change in the position of the puncture target is larger than or equal to the threshold or larger than the threshold, whether or not puncture of the puncture target is possible by controlling the drive unit and changing a movement path of the puncture needle from information regarding a position of a tip of the puncture needle and the position of the puncture target;
change the movement path of the puncture needle if determining that the puncture of the puncture target is possible; and
stop the movement of the puncture needle if determining that the puncture of the puncture target is impossible.

11. The vascular puncture apparatus according to claim 10, wherein the control unit is configured to:
determine, if the amount of change in the position of the tissue including the puncture target is smaller than the threshold or smaller than or equal to the threshold and the amount of change in the position of the puncture target is larger than or equal to the threshold or larger than the threshold, whether or not the position of the tip of the puncture needle has reached a same depth as a rear wall of a blood vessel from the information regarding the position of the tip of the puncture needle and the position of the puncture target;
change the movement path of the puncture needle if determining that the position of the tip of the puncture needle has not reached the same depth as the rear wall of the blood vessel; and
stop the movement of the puncture needle if determining that the position of the tip of the puncture needle has reached the same depth as the rear wall of the blood vessel.

12. The vascular puncture apparatus according to claim 10, wherein the control unit is configured to:
determine, if the amount of change is larger than or equal to the threshold or larger than the threshold, whether or not the tip of the puncture needle is in a body,
stop the movement of the puncture needle by the drive unit if determining that the tip of the puncture needle is in the body; and
stop, if determining that the tip of the puncture needle is not in the body, the movement of the puncture needle by the drive unit and returns the puncture needle to an original position.

13. The vascular puncture apparatus according to claim 1, wherein the control unit is configured to:
calculate the amount of change in the position of the puncture target or the tissue including the puncture target or the amount of change in the position of the vascular puncture apparatus by comparing detection results obtained by the detection unit at any two points in time after the start of the puncture operation by the drive unit; and
stop the movement of the puncture needle by the drive unit or change the movement path of the puncture needle if the amount of change is larger than or equal to the threshold or larger than the threshold.

14. A vascular puncture apparatus comprising:
a detection unit configured to detect at least one of relative positions between the vascular puncture apparatus and a puncture target, relative positions between the vascular puncture apparatus and tissue including the puncture target, a position of the vascular puncture apparatus, and an inclination of the vascular puncture apparatus; and
a control unit configured to:
control movement of a drive unit that includes a puncture needle;
obtain a detection result before puncture from the detection unit before the puncture operation by the drive unit;
continuously or intermittently obtain, after a start of the puncture operation by the drive unit, a detection result after the start of the puncture from the detection unit;
calculate an amount of change in at least one of the relative positions between the vascular puncture apparatus and the puncture target, the relative positions between the vascular puncture apparatus and the tissue including the puncture target, the position of the vascular puncture apparatus, and the inclination of the vascular puncture apparatus by comparing the detection result after the start of the puncture with the detection result before the puncture; and
stop the movement of the puncture needle by the drive unit or changes a movement path of the puncture needle if the at least one amount of change is larger than or equal to a threshold or larger than the threshold.

15. A method for controlling a vascular puncture apparatus including a fixing base including a holding unit capable of holding a part of a human body, a notification unit that notifies of information using an image and/or sound, a drive unit that moves a puncture needle, and a control unit capable of controlling the movement of the drive unit, the method comprising:
obtaining, before a puncture operation by the drive unit, a detection result before puncture using a detection unit capable of detecting at least one of relative positions between the vascular puncture apparatus and a puncture target, relative positions between the vascular puncture apparatus and tissue including the puncture target, a position of the vascular puncture apparatus, and an inclination of the vascular puncture apparatus;
continuously or intermittently obtaining, after a start of the puncture operation by the drive unit, a detection result after the start of the puncture from the detection unit;
calculating an amount of change in at least one of the relative positions between the vascular puncture apparatus and the puncture target, the relative positions between the vascular puncture apparatus and the tissue including the puncture target, the position of the vascular puncture apparatus, and the inclination of the vascular puncture apparatus by comparing the detection result after the start of the puncture with the detection result before the puncture; and
stopping the movement of the puncture needle by the drive unit or changes a movement path of the puncture needle if the at least one amount of change is larger than or equal to a threshold or larger than the threshold.

16. The method according to claim 15, wherein the detection unit includes a cross-sectional image detection portion, which is an echograph that obtains a cross-sectional image, and the method further comprises:
detecting a feature point indicating a specific site from the cross-sectional image and calculate the position of the puncture target or the tissue including the puncture target.

17. The method according to claim 16, wherein the feature point is at least one of a shape of a blood vessel, a contour of a bone, or a contour of a muscle.

18. The method according to claim 15, wherein the detection unit includes an image detection portion, which is a camera that obtains an image, and the method further comprises:
detecting a feature point indicating a specific site from the image and calculate the position of the tissue including the puncture target.

19. The method according to claim 18, wherein the feature point is at least one of a shape of an arm, a position of a vein, size of a lentigo, or a position of a lentigo.

20. The method according to claim 15, wherein the detection unit includes an arm position detection portion that detects a relative distance from the vascular puncture apparatus to an object held by the holding unit, and the method further comprises:
calculating the position of the tissue including the puncture target from the relative distance obtained from the arm position detection portion.
